(19) United States
(12) Patent Application Publication
(10) Pub. No.: US 2026/0188521 A1
(43) Pub. Date: Jul. 2, 2026

(54) ADVANCED TREATMENT PLANNING SYSTEM

(71) Applicant: Milan Madhavji, Mississauga (CA)

(72) Inventor: Milan Madhavji, Mississauga (CA)

(21) Appl. No.: 19/063,693

(22) Filed: Feb. 26, 2025

(30) Foreign Application Priority Data
Dec. 30, 2024 (CA) 3260961

(51) Int. Cl. G16H 50/50; A61B 6/46; A61B 6/51; G16H 30/40

(52) U.S. Cl. CPC G16H 50/50; A61B 6/465; A61B 6/51; G16H 30/40

(57) ABSTRACT

A dental treatment planning system comprises an input module for receiving and accessing dental scan data, a 3D visualization module for generating a 3D virtual model, a report generation module for creating a radiology report with dental findings, a treatment planning module for generating a treatment plan corresponding to the dental findings, and a communication module for presenting the 3D virtual model, radiology report, and treatment plan to the patient using a graphical user interface with interactive navigation and review controls.

The present invention relates to dental treatment planning systems. More specifically, the present invention relates to an advanced treatment planning system that integrates radiological findings with a dentist's treatment plan and patient input.

### BACKGROUND

In the field of dental care, accurate communication of diagnostic information between radiologists, dentists and patients is important for effective treatment planning and for ensuring optimal patient outcomes. Traditional methods of dental imaging and treatment planning often rely on two-dimensional radiographs and manual annotations, which may be time-consuming and prone to errors. With the advent of advanced imaging technologies, three-dimensional (3D) dental scans have become increasingly prevalent, offering a more comprehensive view of the patient's dental anatomy.

Despite the advantages of 3D dental scans, integrating these scans into a cohesive treatment plan remains a significant challenge. Current systems often lack the capability to seamlessly combine radiology reports with the dentist's treatment plans, leading to inefficiencies and a lack of comprehensive care for the patient. This disjointed approach results in miscommunication and delays in treatment, ultimately affecting patient satisfaction and outcomes.

Moreover, there is a notable gap in patient comprehension of dental radiological information and treatment planning. Patients often struggle to understand technical jargon, interpret radiographic images, and grasp the implications of diagnostic findings for their oral health. This lack of understanding hinders their ability to make informed decisions about their treatment options.

Therefore, there is a need for a system that integrates radiological findings, the dentist's treatment plan, and patient input into a single cohesive platform. Such a system would not only streamline the treatment planning process but also enhance patient engagement and understanding, leading to better-informed decisions and improved overall care.

### BRIEF SUMMARY

The present invention relates to dental treatment planning systems. More specifically, the present invention relates to an advanced treatment planning system that integrates radiological findings with a dentist's treatment plan and patient input.

In one aspect, there is provided a system for dental treatment planning including an input module configured to receive and provide access to dental scan data of a dentition of a patient, a 3D visualization module configured to generate a 3D virtual model of the dentition based on the dental scan data, a report generation module configured to generate a radiology report based on the 3D virtual model, the radiology report identifying at least one dental finding, a treatment planning module configured to generate a treatment plan, the treatment plan including at least one treatment option corresponding to the at least one dental finding, and a communication module configured to present the 3D virtual model, the radiology report, and the treatment plan to the patient via a graphical user interface, the graphical user interface comprising user controls that facilitate interactive navigation and review of the 3D virtual model, the radiology report, and the treatment plan by the patient. In one aspect, the report generation module is configured to generate the radiology report based on input from a radiologist, and the treatment planning module is configured to generate the treatment plan based on input from a dentist.

In a further aspect, the at least one dental finding and the at least one treatment option are represented within the 3D virtual model, and wherein the graphical user interface includes a data structure that is linked in data connection with and visually associated with the at least one dental finding and the at least one treatment option. In a still further aspect, the system further includes user-selectable icons in data connection with the data structure, the user-selectable icons selected from at least one of a first user-selectable icon in a first color indicating no defect at the at least one dental finding, a second user-selectable icon in a second color indicating a moderate defect at the at least one dental finding, or a third user-selectable icon in a third color indicating a severe defect at the at least one dental finding.

In one aspect, the user controls are responsive to user input that enables the patient to view and manipulate the 3D virtual model, the user controls selected from at least one of zooming, rotating, panning, or annotating specific areas of interest. In one aspect, the user controls are responsive to user input that enables the patient to select the at least one dental finding and access additional information associated with the at least one dental finding. In one aspect, the user controls are responsive to user input that enables the patient to select the at least one treatment option in the treatment plan and access additional information associated with the at least one treatment option, the additional information including a detailed description of the at least one treatment option, an estimated cost of the treatment option, and/or a timeline associated with the at least one treatment option.

In one aspect, the 3D virtual model comprises a color-coded 3D virtual model to visually represent different data layers of the dentition. In a further aspect, the 3D visualization module includes a 3D model generation sub-module configured to generate a 3D model of the dental scan data, a segmentation sub-module configured to identify and highlight individual dental structures within the 3D model, and an annotation sub-module configured to apply color-coded annotations to the highlighted individual dental structures to generate the color-coded 3D virtual model.

In one aspect, the 3D virtual model, the radiology report, and the treatment plan are presented to the patient as an integrated report via the graphical user interface, the integrated report comprising interactive elements that link the 3D virtual model with the at least one dental finding and the at least one treatment option. In another aspect, the 3D virtual model, the radiology report, and the treatment plan are communicated to the patient via an email containing a link to a secure patient portal.

In a further aspect, the system includes a scheduling module in data communication with the communication module for enabling the patient to schedule an appointment with a dentist, via the graphical user interface, based on the treatment plan. In a still further aspect, the system includes a payment processing module in data communication with the communication module for receiving payment from the patient for viewing and/or implementing the treatment plan.

In another aspect, the user controls include one or more of tools for at least one of rotating, zooming, panning, and resetting a view of the 3D virtual model, interactive 3D manipulation tools enabling the patient to at least one of click, drag, and rotate the 3D virtual model to view it from various angles, dropdown menus or tabs for selecting the at least one dental finding and the at least one treatment option, clickable elements to enable interaction with the treatment plan or accessing additional information, or highlighting or annotation tools for emphasizing areas of interest on the 3D virtual model or providing patient feedback.

In one aspect, there is provided a non-transitory computer-readable medium having instructions stored thereon which, when executed by at least one computer processor, cause the at least one computer processor to receive and provide access to dental scan data of a dentition of a patient, generate a 3D virtual model of the dentition based on the dental scan data, generate a radiology report based on the 3D virtual model, the radiology report including at least one dental finding, generate a treatment plan, the treatment plan including at least one treatment option corresponding to the at least one dental finding, and present the 3D virtual model, the radiology report, and the treatment plan to the patient via a graphical user interface, the graphical user interface comprising user controls that facilitate interactive navigation and review by the patient.

In a further aspect, the at least one dental finding and the at least one treatment option are represented within the 3D virtual model, and wherein the graphical user interface includes a data structure that is linked in data connection with and visually associated with the at least one dental finding and the at least one treatment option. In a still further aspect, non-transitory computer-readable medium includes instructions for displaying user-selectable icons selected from at least one of a first user-selectable icon in a first color, in data connection with the data structure, indicating no defect at the at least one dental finding, a second user-selectable icon in a second color, in data connection with the data structure, indicating a moderate defect at the at least one dental finding, or a third user-selectable icon in a third color, in data connection with the data structure, indicating a severe defect at the at least one dental finding.

In one aspect, the user controls are responsive to user input that enables the patient to at least one of view and manipulate the 3D virtual model, the user controls selected from at least one of zooming, rotating, panning, or annotating specific areas of interest, select the at least one dental finding in the radiology report and access additional information associated with the at least one dental finding, or select the at least one treatment option in the treatment plan and access additional information associated with the at least one treatment option, the additional information including a detailed description of the at least one treatment option, an estimated cost of the treatment option, and/or a timeline associated with the at least one treatment option.

In one aspect, the at least one computer processor is further configured to schedule an appointment with a dentist, via the graphical user interface, based on the treatment plan.

In one aspect, the 3D virtual model comprises a color-coded 3D virtual model to visually represent different data layers of the dentition, and wherein the at least one computer processor is further configured to generate a 3D model of the dental scan data, identify and highlight individual dental structures within the 3D model, and apply color-coded annotations to the highlighted dental structures to generate the color-coded 3D virtual model.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a system in accordance with one aspect;

FIG. 2 illustrates a dental diagnosis and treatment module in accordance with one aspect;

FIG. 3 is a flow chart illustrating a method in accordance with one aspect;

FIG. 4A illustrates a view of an advanced treatment plan in accordance with one aspect;

FIG. 4B illustrates a view of an advanced treatment plan in accordance with one aspect;

FIG. 4C illustrates a view of an advanced treatment plan in accordance with one aspect;

FIG. 5 illustrates a view of a raw dental scan in accordance with one aspect;

FIG. 6 illustrates a view of a web page for accessing an advanced treatment plan in accordance with one aspect;

FIG. 7 illustrates a view of an email for accessing an advanced treatment plan in accordance with one aspect;

FIG. 8 illustrates a view of an advanced treatment plan in accordance with one aspect;

FIG. 9 illustrates a view of an advanced treatment plan in accordance with one aspect;

FIG. 10 illustrates a view of a radiology report in accordance with one aspect; and

FIG. 11 illustrates a view of an advanced treatment plan in accordance with one aspect.

### DETAILED DESCRIPTION

The present invention relates to dental treatment planning systems. More specifically, the present invention relates to an advanced treatment planning system that integrates radiological findings with a dentist's treatment plan and patient input.

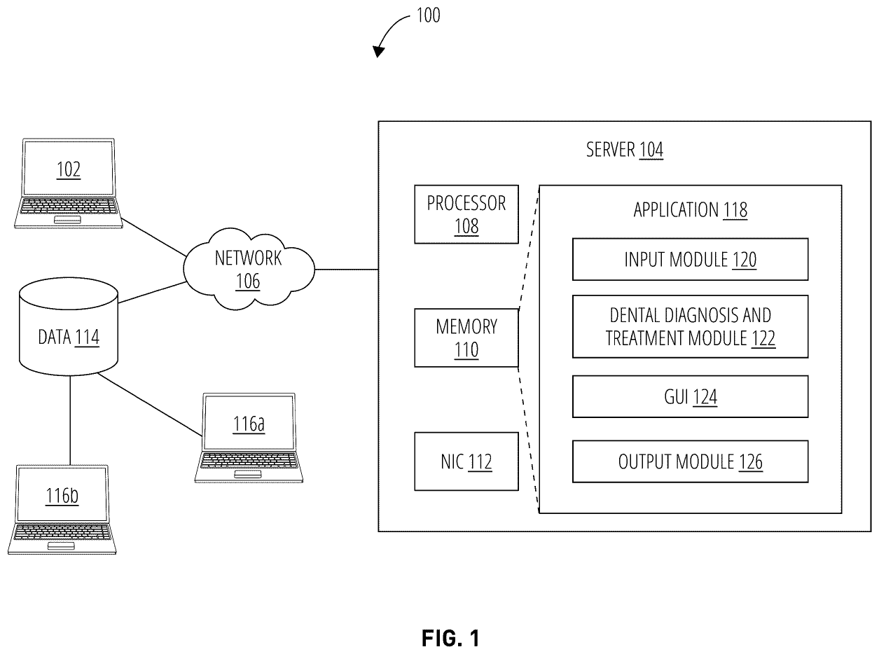

FIG. 1 is a block diagram illustrating an example system 100, according to one aspect. The system 100 includes at least one client device 102 which may comprise one or more computers accessible by one or more users, such as a patient. Client device 102 may be any suitable device, for example, a laptop, a smart phone, a tablet, a wearable device, and so on. Client device 102 may include a memory and a processor for, respectively, storing and executing one or more modules. The memory may include one or more suitable storage media such as a magnetic storage device, a solid-state drive, or random access memory (RAM).

The example aspect of FIG. 1 further includes at least one server 104. In further aspects, server 104 may be implemented as one or more cloud-based servers, such as a cloud-based computing platform. In some aspects, server 104 may perform the functionalities as discussed herein as part of a “cloud” network or may otherwise communicate with other hardware or software components within one or more cloud computing environments to send, retrieve, or otherwise analyze data or information described herein. For example, in aspects of the present techniques, the cloud computing environment may comprise a customer on-premise computing environment, a multi-cloud computing environment, a public cloud computing environment, a private cloud computing environment, and/or a hybrid cloud computing environment.

Client device 102 and server 104 are connected by way of network 106. Network 106 may comprise any suitable network. Generally, the network enables bidirectional communication between the client device 102 and the server 104. Server 104 may include a single, standalone server or may include a plurality of servers in data exchange communication with one another, such as in a server system environment.

In the aspect of FIG. 1, there is also connected to server 104, via network 106, data storage device 114 upon which may be stored data required for operation of the system as described herein or for execution of computing instructions as described herein. Data storage device 114 may have stored thereon patient data, such as dental scan data representing a patient's dentition. The patient data may be accessed or retrieved as needed by server 104 and/or applications or modules stored thereon in response to execution of computer-executable instructions as described herein.

One or more diagnostician systems 116a, 116b may be connected directly with data storage device 114, as shown in the aspect of FIG. 1. In another aspect, diagnostician systems 116a, 116b may be connected with data storage device 114 via network 106. Diagnostician systems 116a, 116b are a system of one or more computers, diagnostic equipment and other components used by a diagnostician for the purpose of generating and analyzing patient data to be stored on data storage device 114 and/or accessed by server 104. In one aspect, diagnostician system 116a is used by a radiologist to generate and analyze patient data and diagnostician system 116b is used by a dentist to generate a treatment plan based on the patient data.

Server 104 may include a processor 108, memory 110, and a network interface controller (NIC) 112. The NIC 112 may include any suitable network interface controller(s), and may communicate over the network 106 via any suitable wired and/or wireless connection. The server 104 may include one or more input device (not depicted) and may include one or more devices for allowing a user to enter inputs (e.g., data) into the server 104. For example, the input device may include a keyboard, a mouse, a microphone, a camera, etc. The NIC may include one or more transceivers that may be used in receipt and transmission of data via external/network ports connected to network.

The processor 108 may include one or more suitable processors (e.g., central processing units (CPUs) and/or graphics processing units (GPUs)). The processor may be connected to the memory 110 via a computer bus (not shown) responsible for transmitting electronic data, data packets, or otherwise electronic signals to and from the processor 108 and memory 110 in order to implement or perform the machine-readable instructions, methods, processes, or elements, as illustrated or described herein. The processor 108 may interface with the memory 110 via a computer bus to execute an operating system (OS) and/or computing instructions contained therein, and/or to access other services/aspects. For example, the processor 108 may interface with the memory 110 via the computer bus to create, read, update, delete, or otherwise access or interact with the data stored in memory 110 and/or the data storage device 114.

The memory 110 may include one or more forms of volatile and/or non-volatile, fixed and/or removable memory. The memory 110 may store an operating system (OS) capable of facilitating the functionalities, applications, methods, computer-executable instructions or software as discussed herein.

The memory 110 may store a plurality of modules, implemented as respective sets of computer-executable instructions as described herein. In the aspect shown in FIG. 1, the plurality of modules is in the form of an application 118. In another aspect, the plurality of modules may be in the form of a plurality of applications or other operations cooperating to produce the set of computer-executable instructions. In general, a computer program or computer based product, application, or code may be stored on a computer usable storage medium, or tangible, non-transitory computer-readable medium (e.g., standard random access memory (RAM), an optical disc, a universal serial bus (USB) drive, or the like), having such computer-readable program code or computer-executable instructions embodied therein, wherein the computer-readable program code or computer-executable instructions may be installed on or otherwise adapted to be executed by the processor 108 (e.g., working in connection with the respective operating system in memory) to facilitate, implement, or perform the machine readable instructions, methods, processes, or elements, as illustrated or described herein.

In the aspect shown in FIG. 1, application 118 includes an input module 120, a dental diagnosis and treatment module 122, a graphical user interface (GUI) 124 and an output module 126.

Input module 120 includes a set of computer-executable instructions for implementing communication functions. The input module 120 may include a communication component configured to communicate (e.g., send and receive) data via one or more external/network port(s) to one or more networks or local terminals, such as network 106, the client device 102, and/or diagnostician systems 116a, 116b described herein. In some aspects, server 104 may include a client-server platform technology responsible for receiving and responding to electronic requests. In one aspect, the input module 120 is responsible for receiving and providing access to raw dental scan data of a patient's dental anatomy from the data storage device 114 through network 106.

Embedded within or accessible to the application 118 is a dental diagnosis and treatment module 122. Dental diagnosis and treatment module 122 processes and analyzes the raw dental scan data of a patient's dental anatomy received from the input module 120. Dental diagnosis and treatment module 122 includes advanced imaging software designed to create, edit, process, analyze, and display images. This type of software can handle various image formats from simple photographs to complex graphics and medical scans. Imaging software may include one or more machine learning models or an artificial intelligence system capable of performing automated image analysis, accessible by server 104.

Dental diagnosis and treatment module 122 is configured to receive data inputs via the input module 120. Such data inputs may include an image acquired from a scanning device or images stored in data storage device 114 or elsewhere that is accessible by the dental diagnosis and treatment module 122. In one aspect, dental diagnosis and treatment module 122 includes medical imaging software used to manage and process images obtained from various diagnostic tools such as X-rays, CT scans, MRIs, ultrasound, and other imaging modalities. In one aspect, the patient image data may be in a DICOM (Digital Imaging and Communications in Medicine) file, which is a specialized file format widely used in the field of medical imaging. DICOM files typically contain medical images, such as image data from medical or dental scanning operations. DICOM files may be used in advanced visualization techniques, such as 3D reconstruction from scan data. DICOM files may also include metadata. Metadata may contain information about the patient (e.g., name, ID, date of birth), imaging procedure (e.g., type of scan, parameters), and equipment used (e.g., device manufacturer, settings). Metadata makes the DICOM file self-contained and useful for clinical and diagnostic purposes. DICOM also includes a communication protocol that allows devices, such as imaging scanners, workstations, and PACS (Picture Archiving and Communication Systems), to exchange files seamlessly. Physicians and radiologists may use DICOM files to view and analyze diagnostic images for various conditions.

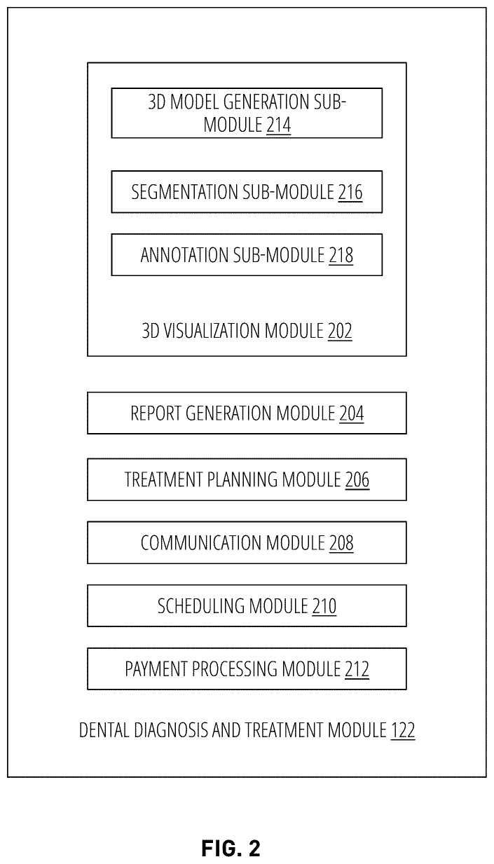

Dental diagnosis and treatment module 122 accesses data, via input module 120, from data storage device 114, client device 102 or diagnostician systems 116a, 116b as required. In one aspect, data which is accessed by dental diagnosis and treatment module 122 includes the dental scan data of a patient's dentition. The dental diagnosis and treatment module 122 processes the dental scan data and presents it via the GUI 124, enabling diagnosticians to review and refine reports and treatment plans using the diagnostician systems 116a, 116b and allowing patients to access and engage with their reports and treatment plans through the client device 102. Dental diagnosis and treatment module 122 enables real-time communication and collaboration between the radiologist, the dentist and the patient, ensuring that all relevant information is included and communicated effectively through the treatment plan generated by the module. Dental diagnosis and treatment module 122 is discussed in further detail with respect to FIG. 2.

GUI 124 facilitates user interaction and visualization capabilities within the system 100. GUI 124 includes a set of computer-executable instructions, which, when executed by at least one processor, cause the processor to couple or connect GUI 124 with a 3D virtual model of a patient's dentition, a radiology report and/or a treatment plan generated by the dental diagnosis and treatment module 122 and provide one or more controls by which a user, including a radiologist, a dentist, or a patient, may make user inputs for the purpose of viewing or updating the radiology report and treatment plan.

GUI 124 provides a visual interface that enables users to interact with the system, offering a dynamic, visually intuitive platform for managing treatment plans. GUI 124 includes interactive elements, such as buttons, menus, sliders, text boxes, and so on, that enhance functionality and user engagement. For diagnosticians, it features tools such as zoomable 3D dental models, annotation capabilities, drag-and-drop treatment adjustments, and overlays for comparing pre-and post-treatment simulations. Patients interact with simplified elements like touch-friendly sliders to explore treatment options, pop-up explanations for procedures, and interactive calendars for scheduling or confirming appointments. Additional features include user-selectable or clickable icons for accessing treatment summaries, progress tracking charts, and forms for submitting feedback or preferences. The GUI 124 adapts across devices, ensuring seamless interaction on both professional diagnostic systems and patient-facing devices like smartphones and tablets. The GUI 124 simplifies complex processes and fosters collaboration and informed decision-making. In one aspect, GUI 124 provides a diagnostician with a detailed, technical view of the processed dental scan data, complete with advanced tools for analysis, simulation, and treatment planning refinement. In one aspect, GUI 124 simplifies complex information into clear, visually engaging formats, enabling patients to review treatment plans, provide feedback, and make informed decisions.

Output module 126 serves various functions within the system 100. In one aspect, output module 126, upon receiving instructions from GUI 124, generates and delivers formatted, user-ready radiology reports and treatment plans for a patient's perusal. The output module 126 may be a standalone display or may be a display that is integrated or coupled with client device 102 so that the radiology report and treatment plan are displayed to a user. In some aspects, the output module 126 is configured to incorporate GUI 124 wherein the output module 126 and GUI 124 may operate collectively.

For diagnosticians, output module 126 produces detailed reports, printable 3D models, and charts summarizing key diagnostic metrics or treatment outcomes. For patients, the output module 126 generates simplified, visually appealing treatment summaries, downloadable PDFs, and appointment schedules. Output module 126 ensures consistency in formatting, adhering to predefined templates and branding requirements, while also accommodating various delivery formats such as on-screen displays, printable documents, or shareable files. In one aspect, the output module 126 can securely share treatment plans via email links. Once a treatment plan is finalized, the output module 126 uploads the treatment plan to an online cloud system, generates an encrypted link to the report and then sends the encrypted link to the patient via email. Patients may then access their treatment details through a secure, time-sensitive portal. This ensures both confidentiality and convenience, enabling patients to review their plans on any device while adhering to data privacy regulations. In another aspect, the link may direct the patient to a web page having a link which may allow a user to enter the portal. Operating as a non-interactive component, the output module 126 integrates seamlessly with the system's other modules and GUI 124, ensuring that the generated content is ready for secure sharing or real-time presentation.

As shown in FIG. 2, dental diagnosis and treatment module 122 includes a plurality of modules that include computer-executable instructions for execution by at least one processor. In the aspect shown in FIG. 1, the plurality of modules cooperates to provide functionality and operability to the dental diagnosis and treatment module 122. The dental diagnosis and treatment module 122 comprises a 3D visualization module 202, a report generation module 204, a treatment planning module 206, a communication module 208, a scheduling module 210, and a payment processing module 212, each serving specific roles in facilitating a comprehensive and efficient dental treatment planning process.

The 3D visualization module 202 is configured to generate a 3D virtual model of a patient's dentition based on the raw dental scan data of the patient that is accessible through the input module 120 (shown in FIG. 1). 3D visualization module 202 processes the raw dental scan data to produce a highly detailed virtual model of a patient's dentition. The 3D virtual model may be segmented and annotated or may be designed to enhance clarity and ease of reference. In one aspect, the 3D visualization module 202 generates a 3D virtual model of a patient's dentition which may be rendered in color. In a preferred aspect, the 3D visualization module 202 generates a color-coded 3D virtual model of the patient's dentition. The term “color-coded” refers to the application of distinct colors to visually represent various features, attributes, or data layers of the 3D virtual model. For instance, it may highlight dental conditions (e.g., red for cavities, blue for healthy teeth, or yellow for areas of concern), differentiate anatomical structures (e.g., gum tissue, enamel, or bone), or illustrate treatment recommendations (e.g., green for areas requiring monitoring, orange for moderate priority, or red for urgent intervention). Additionally, the color-coding may reflect specific data layers for diagnostic metrics, such as bone density, gum health, or implant placement. The term may also encompass a proprietary color scheme uniquely tailored to segment and identify different sections of the dentition, such as molars, premolars, canines, and incisors, enabling more precise analysis and communication. The use of color-coding provides a clear and intuitive means for users to identify key findings and navigate the model with ease. It should be understood that the term “color-coded” does not imply a strict requirement that color be used. For example, the color-coded 3D virtual model may be rendered with greyscale coloration or other shading or hatching to distinguish between features, attributes or data layers.

In one aspect, 3D visualization module 202 includes several sub-modules such as a 3D model generation sub-module 214, a segmentation sub-module 216, and an annotation sub-module 218. The 3D model generation sub-module 214 initiates the process by converting the dental scan data into a 3D virtual model, providing a comprehensive view of the patient's dental anatomy. The 3D model generation sub-module 214 dynamically adjusts, preferably in real time, various visual elements such as opacity, brightness, contrast, color balance, sharpness, and scaling. These adjustments may be tailored to optimize the appearance of the 3D model based on user inputs or contextual factors arising from interactions with the GUI 124. In one aspect, this adjustment process may be automated using algorithms that analyze the 3D model and user behavior within the GUI 124 to refine settings for better visibility, clarity, or aesthetic appeal. For example, the sub-module may enhance contrast to highlight critical findings or adjust color balance to emphasize particular features of interest. By dynamically adjusting image attributes, the 3D model generation sub-module 214 significantly enhances the user experience, allowing patients, dentists, and radiologists to interact with the virtual model in an intuitive and context-sensitive manner. This adaptability ensures that the visual presentation of the dentition is optimized for diverse situations, such as patient consultations, diagnostic evaluations, or treatment planning sessions, thereby improving both the accessibility and efficacy of the system.

The segmentation sub-module 216 is responsible for identifying and highlighting individual dental structures within the 3D virtual model. In this context, highlighting encompasses various methods such as coloring, shading, outlining, or using other visual cues to emphasize and/or visually distinguish individual dental structures within the 3D model. In one aspect, segmentation sub-module 216 utilizes advanced algorithms to automatically or semi-automatically distinguish and isolate different anatomical structures within the dental scans, such as teeth, roots, bones, nerves, and soft tissues. This segmentation enables the creation of detailed, high-resolution visualizations that assist dental professionals in diagnosis, treatment planning, and surgical simulations. The segmentation sub-module 216 may include a user-friendly interface that allows dental professionals to refine and adjust the segmentation to account for individual anatomical variations, thereby ensuring the accuracy and effectiveness of dental treatments. In a further aspect, the segmentation sub-module 216 integrates machine learning algorithms to automate the detection and identification of pathological areas, such as cavities, fractures, or bone density loss, facilitating early diagnosis and intervention. This module further includes coloration capabilities, automatically assigning distinct colors to segmented structures to enhance visualization. For instance, teeth, bones, nerves, and soft tissues can be displayed in different colors, making it easier for dental professionals to differentiate between these structures. Different types of teeth—such as molars, premolars, canines, and incisors—can also be highlighted in unique colors within the segmented 3D model, contributing to a comprehensive and color-coded virtual representation. This advanced visualization significantly improves the user's ability to assess the patient's dental anatomy, identify issues, and plan treatments with precision and confidence.

The annotation sub-module 218 works in conjunction with the segmentation sub-module 216 to enhance the utility and interpretability of the 3D virtual model. This sub-module applies additional color-coded highlights and textual annotations to the segmented structures, incorporating visual aids such as borders, icons, buttons, or other markers to convey the severity or nature of detected issues. In one aspect, the color-coded annotations may include red icons for severe issues, yellow icons for moderate concerns, and green icons for healthy areas in the color-coded 3D virtual model. These color-coded annotations streamline the communication of diagnostic insights by making critical information immediately visible and easily understood.

In one aspect, the annotation sub-module 218 allows manual annotations by diagnosticians, allowing them to add personalized comments, observations, or recommendations directly to the 3D virtual model. This feature enables tailored insights based on the clinician's expertise and the specific needs of the patient. Additionally, the annotation sub-module 218 integrates seamlessly with the system's user interface, ensuring that annotations are accessible and editable in real time, providing flexibility during consultations or treatment planning sessions.

Together, each of the sub-modules within the 3D visualization module 202 collaborate to generate a detailed and interactive color-coded 3D virtual model of a patient's dentition, providing a precise representation of their dental anatomy and health.

The report generation module 204 is configured to generate a detailed radiology report based on the color-coded 3D virtual model created by the 3D visualization module 202. This module compiles a comprehensive summary of the highlighted and annotated regions within the color-coded 3D virtual model, identifying relevant dental findings or conditions. These summaries may include textual descriptions, severity indicators, and color-coded representations, all integrated into a cohesive radiology report. The radiology report serves as a thorough diagnostic tool, facilitating communication between healthcare providers and enhancing patient understanding. By directly incorporating annotations and highlights from the color-coded 3D virtual model, the report generation module 204 ensures that key findings are accurately documented and effectively communicated, supporting improved diagnostic precision, treatment planning, and patient engagement. In one aspect, the report generation module 204 incorporates inputs from a radiologist, further ensuring accuracy and reliability of the dental findings in the radiology report.

The treatment planning module 206 combines diagnostic insights from the report generation module 204 with treatment recommendations to address the dental findings or conditions identified in the radiology report. The treatment planning module 206 is configured to update the radiology report with one or more treatment options to generate a comprehensive and actionable treatment plan. In one aspect, the treatment planning module 206 utilizes advanced algorithms to recommend treatment options tailored to the diagnostic data, ensuring precision and relevance. This module also facilitates direct input from dentists, enabling the customization of treatment plans to meet the specific needs of each patient. Additionally, the treatment planning module 206 may incorporate external data sources, such as patient medical histories and prior treatments, to ensure a personalized treatment strategy. The treatment planning module 206 provides a collaborative platform that enables seamless communication and coordination between radiologists and dentists. By consolidating all relevant diagnostic information and treatment recommendations into a single system, the module ensures that the final treatment plan is both thorough and aligned with the patient's needs and clinical goals.

The communication module 208 is designed to present the color-coded 3D virtual model, the radiology report, and the treatment plan to the patient in an easily understandable format. This module utilizes an interactive interface to present the treatment options, allowing the patient to explore and understand the proposed treatments. The communication module 208 utilizes features such as color coding and visual aids to differentiate between various elements of the treatment plan, emphasizing the severity of identified issues and prioritizing necessary interventions. In one aspect, the module combines the color-coded 3D virtual model, the radiology report, and the treatment plan into an integrated report that seamlessly merges the diagnostic data, visual representations, and treatment recommendations into a single, comprehensive document.

Additionally, the communication module 208 collaborates with the output module 126 to facilitate secure sharing of the treatment plan. Patients receive an email containing a link to a secure patient portal that provides access to their treatment details, leave comments, and ask questions. To further support decision-making, the module may provide educational resources, including interactive diagrams, animations, video tutorials, articles, and frequently asked questions (FAQs), tailored to the specifics of the treatment options. Additionally, the module may incorporate a decision support tool that helps patients compare the benefits, risks, and potential outcomes of different treatment options, empowering them to make informed decisions.

The communication module 208, in conjunction with the GUI 124, focuses on delivering the color-coded 3D virtual model, radiology report, and treatment plan or an integrated report incorporating all three to the patient in an accessible and engaging manner. The module generates interactive presentations, including 3D models, graphical summaries and clickable icons, offering patients a clear visualization of their dental health and recommended treatments. Intuitive elements, such as color-coded markers and interactive icons, guide patients through the treatment plan, ensuring critical information is conveyed effectively. Communication module 208 bridges the gap between healthcare providers and patients, enhancing understanding and engagement throughout the treatment process.

The communication module 208 manages the display of graphical elements and visual components within the GUI 124, to enhance user interaction and navigation. These graphical elements, such as interactive controls, visual indicators, and labels, are designed to enable users to navigate and interact with the radiology report, treatment plan, and color-coded 3D virtual models effectively. The module ensures that all graphical elements are rendered accurately and updated in real-time, adapting to user inputs and dynamic data changes to maintain a responsive and user-friendly interface.

Additionally, these indications may be color-coded to convey the severity of the dental issue associated with the selected region. For instance, red may indicate critical issues requiring immediate attention, yellow may highlight moderate concerns, and green may signify healthy areas. By combining intuitive visual elements with interactive feedback, the communication module 208 enhances user engagement and ensures that both patients and clinicians can efficiently navigate and interpret the information provided by the system 100.

The scheduling module 210 streamlines the appointment scheduling process, allowing patients to view, confirm, and schedule appointments directly based on the proposed treatment plan. In one aspect, scheduling module 210 integrates with the communication module 208 and GUI 124 to provide a seamless experience for the patient. Scheduling module 210 integrates with real-time calendars and/or synced calendars to display the availability of relevant professionals and facilities, enabling patients to select their preferred time slots directly through the GUI 124. The module also automates the sending of notifications and reminders via email or SMS, significantly reducing the likelihood of missed appointments. Furthermore, it synchronizes with the communication module 208 to provide immediate scheduling options as patients review their treatment plans, ensuring an efficient experience.

Payment processing module 212 facilitates secure and efficient financial transactions related to the treatment plan. In one aspect, it supports multiple payment methods, including credit cards, debit cards, and online platforms, and generates receipts and payment confirmations for the patient's records. The payment processing module 212 integrates with the communication module 208 and the scheduling module 210 to provide a smooth and cohesive patient experience.

Collectively, these modules create an integrated workflow within the dental diagnosis and treatment module 122 from diagnosis to treatment, enhancing collaboration among healthcare providers and delivering a user-friendly and comprehensive experience for patients. By automating key processes such as scheduling, notifications, and payments, the system significantly improves operational efficiency while ensuring a high standard of patient satisfaction. This comprehensive approach provides a robust framework for modern dental treatment planning, promoting better outcomes for both patients and healthcare providers.

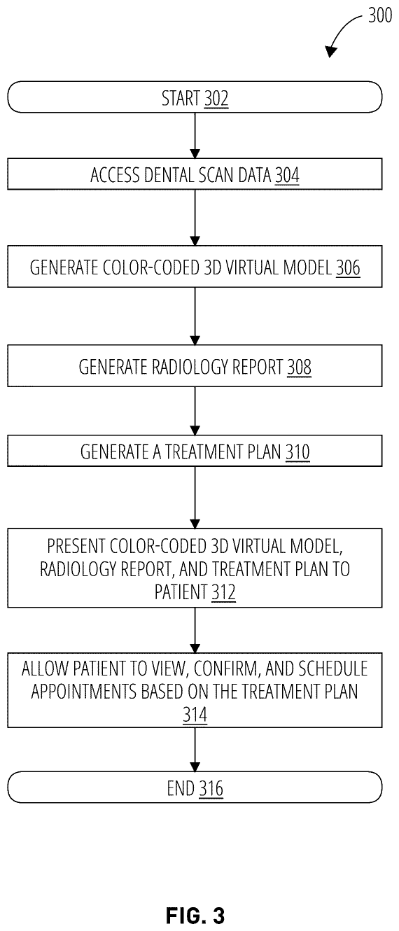

FIG. 3 illustrates a method 300. Preferably, at least one non-transitory computer-readable medium has stored thereon a set of instructions which, when executed by at least one computer processor, cause the at least one computer processor to execute the steps of method 300. The at least one processor may be one or more of the processors of system 100.

The method 300 begins at block 302. The method begins with the activation of the necessary computational and system resources. At this stage, the system 100 validates its readiness by initializing connected modules, including the input, processing, and output modules. System 100 ensures that the network 106 and data storage device 114 are securely connected, enabling the seamless transfer and retrieval of patient-specific dental scan data.

At block 304, the dental scan data of a patient is accessed. In one aspect, the system 100 retrieves the patient's dental scan data from data storage device 114, shown in FIG. 1. The access is facilitated through the input module 120, which uses secure protocols to verify data integrity and prevent unauthorized access. The 3D visualization module 202 accesses this data via the input module 120. Specific protocols, such as secure data transmission and validation checks, may be used to ensure the accuracy and integrity of the retrieved dental scan data.

At block 306, a color-coded 3D virtual model is generated from the dental scan data of the patient. The 3D visualization module 202 processes the accessed dental scan data to generate a color-coded 3D virtual model of the patient's dentition. The 3D visualization module 202 employs several sub-modules including a 3D model generation sub-module 214 for generating a detailed 3D model of the patient's dental anatomy, a segmentation sub-module 216 for identifying individual dental structures and highlighting pathological areas for early detection of potential issues like decay, fractures, or infections, and an annotation sub-module 218 that integrates with the segmentation outputs to apply color-coded highlights, borders, and textual annotations. The generated color-coded 3D virtual model serves as a guide for subsequent diagnosis and treatment planning steps.

At block 308, a comprehensive radiology report is generated based on the color-coded 3D virtual model. The report generation module 204 processes the color-coded 3D virtual model of the patient's dentition to produce a comprehensive radiology report with diagnostic insights. The generated color-coded 3D virtual model and radiology report serve as a guide for subsequent treatment planning steps. In one aspect, the radiology report is produced with input provided by a radiologist.

At block 310, the treatment planning module 206 combines diagnostic insights from the radiology report with treatment recommendations to create a comprehensive treatment plan. Advanced algorithms can be used to analyze the diagnostic data to suggest tailored treatment options, ensuring relevance and accuracy. Treatment planning module 206 allows dentists to customize the plan based on professional judgment and patient-specific factors, such as medical history and prior treatments. This step facilitates real-time collaboration between healthcare providers, ensuring the treatment plan is cohesive and actionable. In one aspect, the treatment plan integrates the radiology report with one or more treatment options, categorized by priority and urgency, to aid decision-making.

At block 312, the communication module 208 presents the color-coded 3D virtual model, radiology report, and treatment plan to the patient in an accessible and engaging format. The color-coded 3D virtual model, radiology report, and treatment plan are displayed to the patient via the GUI 124. In one aspect, a single integrated report that combines the color-coded 3D virtual model, radiology report, and treatment plan may be presented to the patient via the GUI 124. The present format may include clickable elements or user-selectable icons in the color-coded 3D virtual model to indicate severity of any dental findings or conditions, and graphical summaries of each identified dental finding and the recommended treatment options. GUI 124 includes user controls that allow the patient to view and manipulate the color-coded 3D virtual model. The user controls further enable the patient to view the radiology report and explore the one or more treatment options laid out in the treatment plan. In one aspect, the user controls enable the patient to select one of the treatment options and access additional information associated with the selected treatment option. Embedded resources, such as videos and FAQs, provide context to help patients grasp the implications of the proposed treatments. This step prioritizes patient understanding and involvement in the decision-making process.

In one aspect, the user controls include one or more of tools for rotating, zooming, panning, and resetting a view of the color-coded 3D virtual model, sliders for adjusting the color intensity or transparency of specific regions in the model, interactive 3D manipulation tools enabling the patient to click, drag, or rotate the color-coded 3D virtual model to view it from various angles, dropdown menus or tabs for selecting treatment options or toggling between different views of the dentition, clickable elements to enable interaction with the treatment plan, including selecting a treatment option or accessing additional information, and highlighting or annotation tools for emphasizing areas of interest on the color-coded 3D virtual model or providing patient feedback. In another aspect, the user controls may include on-screen graphical controls such as buttons, sliders, and other interactive elements for controlling display of the color-coded 3D virtual model and GUI 124.

In one preferred aspect, the system is configured to enable a user to interact with the color-coded 3D virtual model of a patient's dentition using an input device such as a mouse, where different manipulations, such as rotation, panning, and zooming, may be performed based on specific input actions. These controls provide an invisible and unobtrusive interface configured to allow a user, such as a patient, to focus entirely on the visualization of the color-coded 3D virtual model.

In one preferred aspect, to rotate the color-coded 3D virtual model, the user may hold the left mouse button while moving the mouse in the desired direction of rotation. This input is translated into corresponding rotational adjustments of the color-coded 3D virtual model and GUI 124 around its axes. Panning the color-coded 3D virtual model and GUI 124 may be accomplished by holding the right mouse button and moving the mouse, enabling translation of the color-coded 3D virtual model and GUI 124 along the x-and y-axes. Zooming in and out may be achieved by rotating the mouse wheel, which adjusts the scale of the color-coded 3D virtual model and GUI 124 incrementally or decrementally to bring areas of interest into or out of focus or to increase or decrease their relative scale. By combining these actions, system 100 offers a fluid and natural interaction model for exploring complex image datasets without the need for obtrusive on-screen graphical controls.

In one aspect, the treatment plan communicated to the patient via the GUI 124, includes color-coded annotations that indicate the severity of a defect in the dentition. The color-coded annotations include user-selectable icons or other clickable elements in different colors, for instance, green user-selectable icons indicating a region with no defect where no treatment is recommended, yellow user-selectable icons indicating a region with moderate defects where non-urgent treatment is recommended; and red user-selectable icons indicating a region with severe defects where urgent treatment is recommended.

In one aspect, the communication module 208 works in cooperation with the output module 126 to generate encrypted links for patients to access their treatment plans through a secure, patient portal, adhering to data privacy regulations. This step ensures that the patient is well-informed and empowered to take the next steps in their treatment journey.

Once the treatment plan is communicated, the patient gains access to additional interactive features, enabling further engagement at block 314. The patient is given the ability to plan and schedule appointments based on the treatment plan. In one aspect, the patient has the option to either accept the recommended treatment plan or to request a consult with the dentist to further discuss the treatment plan. The scheduling module 210 enables patients to view real-time availability of healthcare professionals and facilities through an interactive interface. The module also allows patients to leave comments or request adjustments to their treatment plans directly through the GUI 124, fostering an open and collaborative approach. In one aspect, the scheduling module 210 enables patients to directly select and confirm their preferred time slots using the GUI 124. Notifications and reminders are automatically sent via email or SMS to reduce the likelihood of missed appointments. The scheduling module 210 seamlessly integrates with the communication module 208, allowing patients to schedule appointments as they review their treatment plans. The payment processing module 212 facilitates secure, convenient, and flexible payment options. The module enables patients to pay using credit cards, debit cards, or online payment platforms. Transactions are encrypted to ensure security and compliance with data privacy regulations. Receipts and payment confirmations are automatically generated and sent to patients for their records. The payment module integrates with the scheduling module to streamline the process, allowing patients to complete payments during the scheduling of their appointments.

At block 316, the method 300 concludes with a final review of all inputs and outputs. System 100 ensures that all user inputs and payments have been processed and recorded. The output module 126 generates and formats the finalized treatment plan for storage and distribution. Any residual data is securely stored or transmitted as required, adhering to relevant data privacy and security standards.

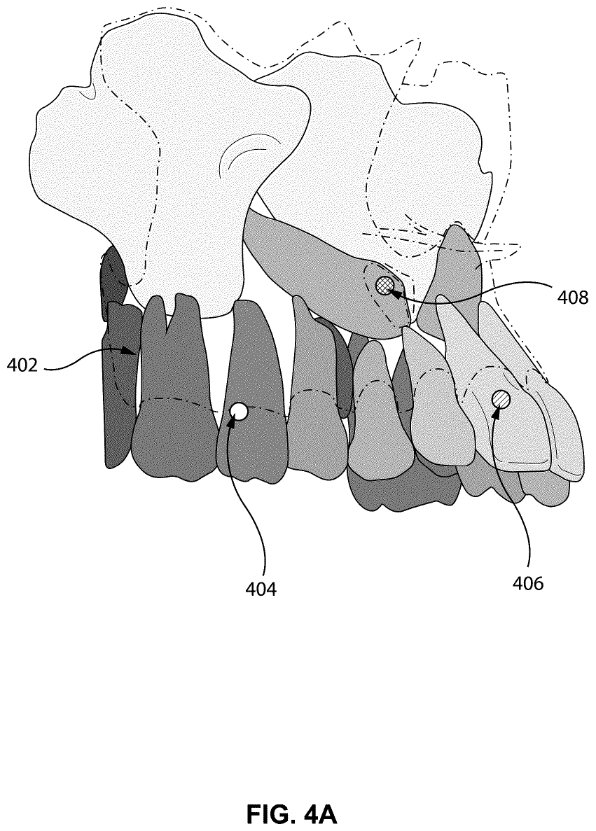

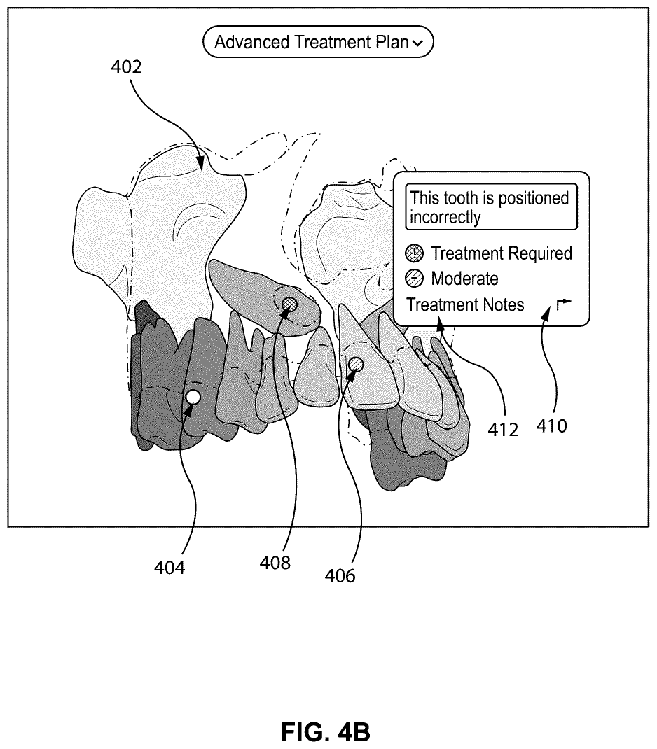

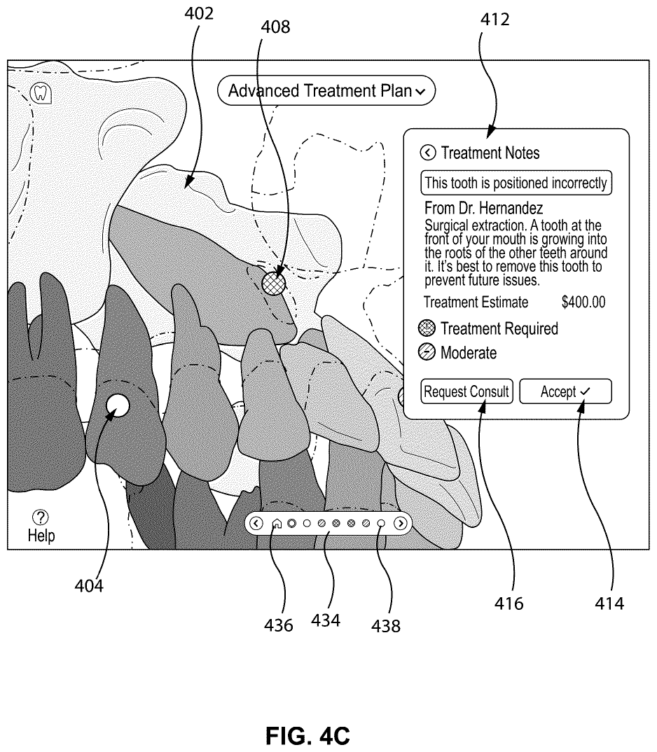

FIG. 4A to FIG. 11 illustrate example outputs and/or graphical representations of the color-coded 3D virtual model, radiology report and treatment plan generated in accordance with the method 300 of FIG. 3 and presented to the patient via GUI 124. For instance, FIGS. 4A to 4C illustrate three different views of a color-coded 3D virtual model with user-selectable icons as presented to a patient via GUI 124.

FIG. 4A illustrates a first view of the color-coded 3D virtual model 402 of a patient's dentition. The color-coded 3D virtual model 402 incorporates user-selectable icons in different colors to indicate various dental findings in the color-coded 3D virtual model. In one aspect, green user-selectable icon 404 identifies a healthy region where no treatment is required. A yellow user-selectable icon 406 identifies a region that may be monitored and where non-urgent treatment is recommended. A red user-selectable icon 408 highlights a problem area where urgent treatment is recommended. The green user-selectable icon 404, yellow user-selectable icon 406 and red user-selectable icon 408 are interactive buttons that reveal additional information upon user interaction, as further illustrated in FIG. 4B.

FIG. 4B illustrates a second view of the color-coded 3D virtual model 402 of FIG. 4A. In this view, a user has clicked on the yellow user-selectable icon 406 to trigger the display of an additional information tab 410 which reveals additional information associated with the region. The additional information tab 410 identifies the defect, for example, the incorrect positioning of a tooth in this instance, and indicates the urgency of treatment for correcting the defect, which is categorized as moderate in this case. The additional information tab 410 also includes an interactive button labelled treatment notes 412 that provides further insights on the recommended treatment plan.

FIG. 4C illustrates a third view of the color-coded 3D virtual model 402 of FIG. 4B, expanding upon the interaction illustrated in FIG. 4B. In this view, a user has clicked the treatment notes 412 button to access additional notes on the recommended treatment plan. For instance, in this example, the recommended treatment plan involves surgical extraction. The additional notes also provide an estimated cost for the recommended treatment plan. At this stage, the patient is presented with actionable options to either accept the recommended treatment plan by clicking the accept 414 button or to request a consult with the dentist by clicking the request consult 416 button.

The view also includes a navigation panel 434 with on-screen graphical controls in the form of different user-selectable icons. These user-selectable icons include a home button 436, which provides a convenient shortcut to the home page, and one or more dental finding buttons 438, which navigate the user to the corresponding dental finding display. The dental finding button 438 may be color-coded, with the color representing the severity level of the associated dental finding, thereby allowing users to quickly identify and prioritize critical findings.

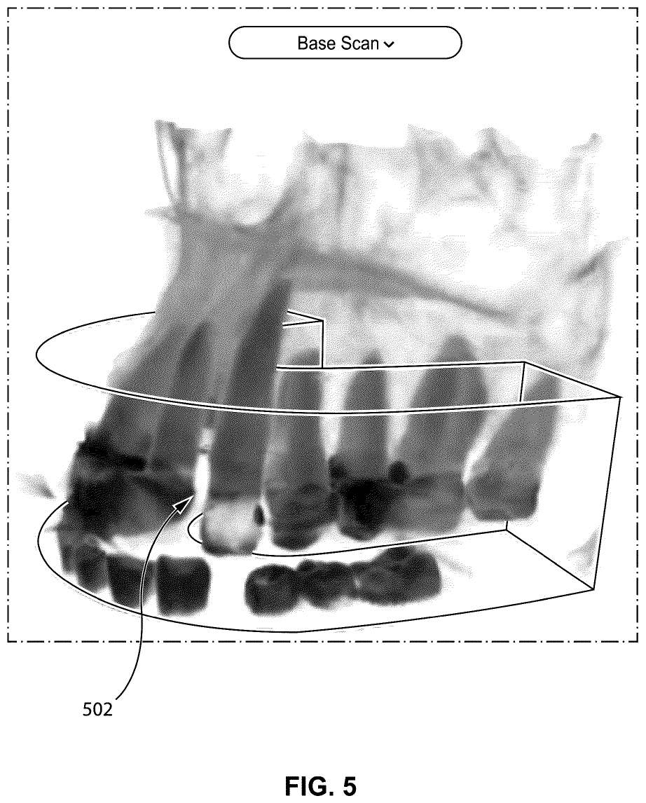

FIG. 5 illustrates a graphical representation of raw dental scan data 502 of a patient's dental anatomy as accessed by the dental diagnosis and treatment module 122 via the input module 120.

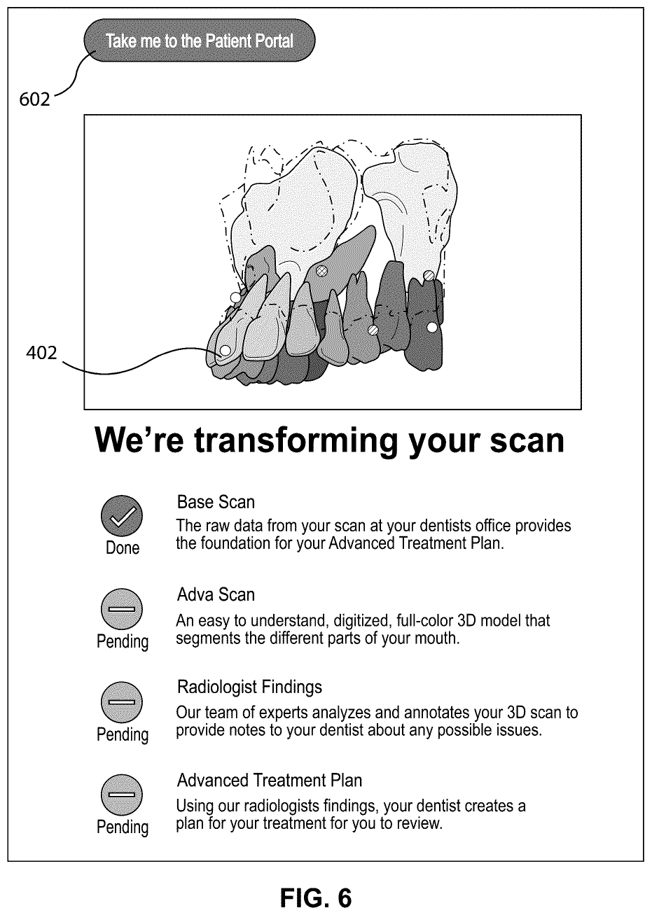

FIG. 6 illustrates an exemplary web page which a patient may access for the purpose of accessing their advanced treatment plan. The web page has a link in the form of a button which allows the patient to access patient portal 602. Within patient portal 602, the patient may view the advanced treatment plan with GUI 124 according to one aspect. Accordingly, via the web page, a patient may gain access to a patient portal 602 for the patient to see the color-coded 3D virtual model 604, radiology report, and treatment plan.

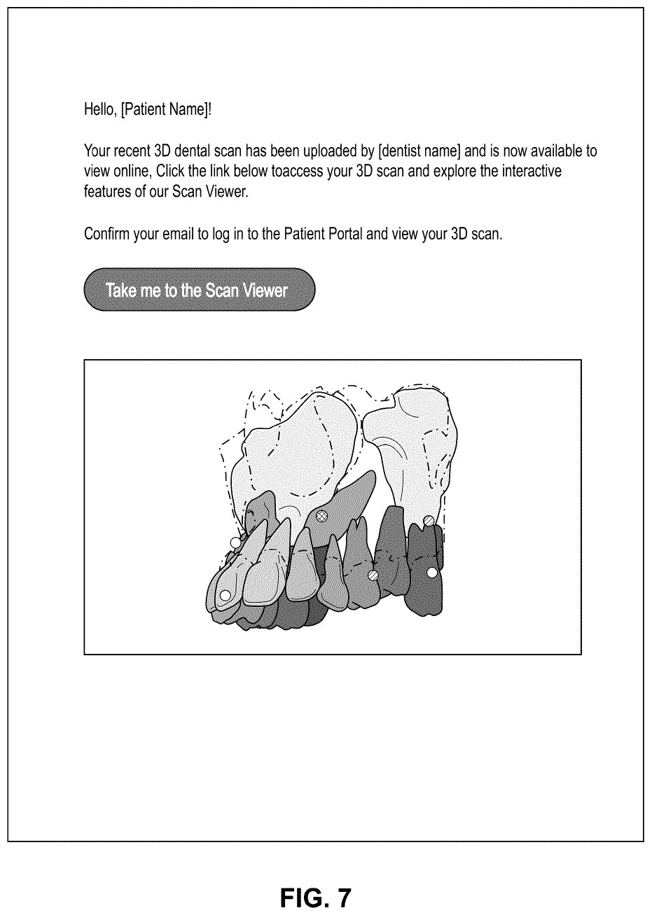

FIG. 7 illustrates an exemplary email sent by output module 126 containing a link which allows the patient to access patient portal 602. Within patient portal 602, the patient may view the advanced treatment plan with via GUI 124, according to one aspect. The email informs the patient that the advanced treatment plan of the patient's dentition is ready for viewing by the patient and provides the patient access to view the advanced treatment plan.

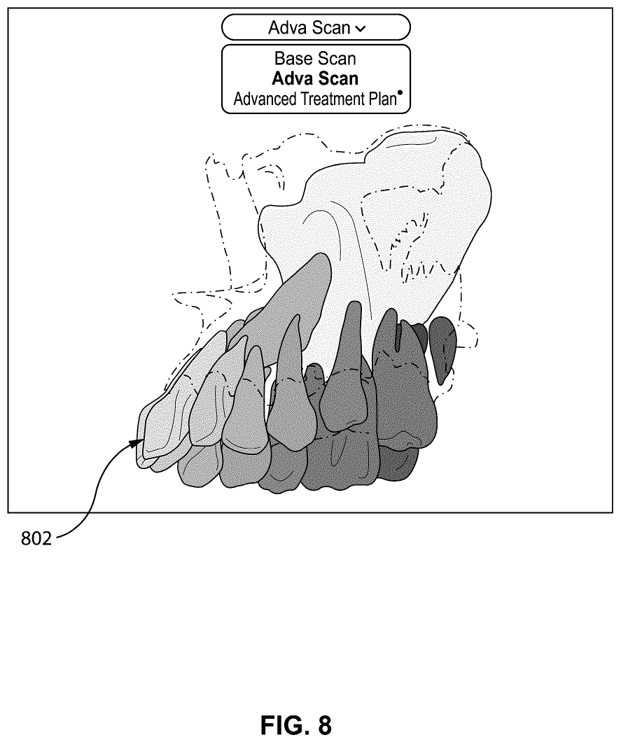

FIG. 8 illustrates a view of the advanced treatment plan showing a color-coded 3D virtual model 802 as presented to the patient, in one aspect.

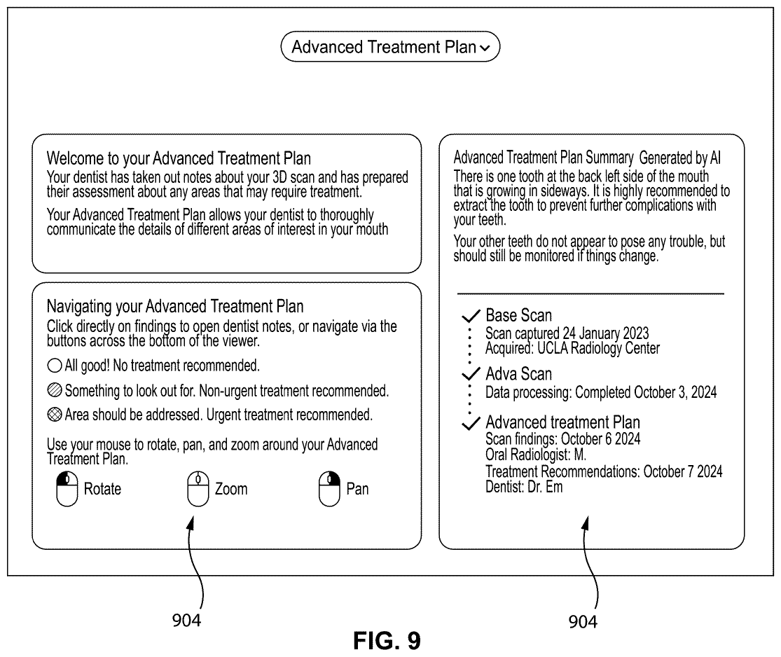

FIG. 9 illustrates a view of the advanced treatment plan as presented to the patient via GUI 124, in one aspect. The view includes a report guide 902 to help the patient to navigate through the advanced treatment plan and a treatment plan summary 904 that provides a brief summary of the advanced treatment plan.

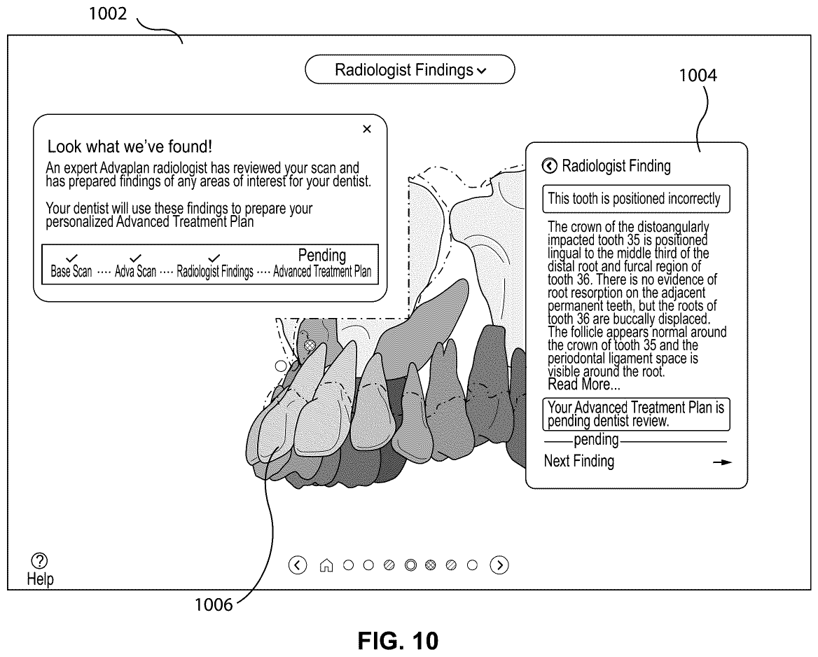

FIG. 10 illustrates a view of a radiology report 1002 as presented to the patient via GUI 124, in one aspect. The view shows a color-coded 3D virtual model 1006 of the patient's dentition along with a dental finding 1004 identified in the radiology report. The patient can review the information provided in the dental finding 1004 and then proceed to see the recommended treatment plan or another dental finding identified in the radiology report 1002.

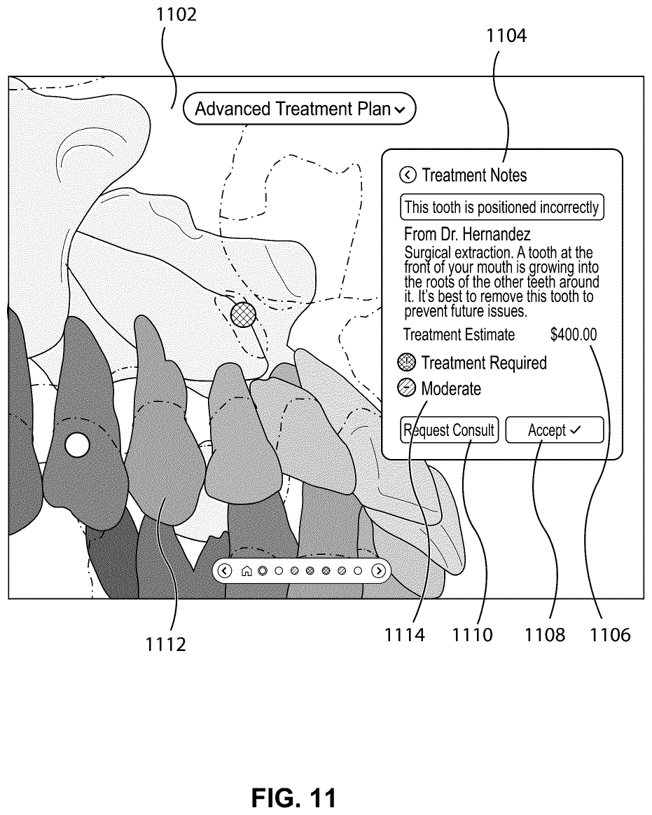

FIG. 11 illustrates a view of a treatment plan 1102 as presented to a patient via GUI 124, in one aspect. The view shows treatment notes 1104 and treatment estimate 1106 for treating the dental finding 1004 identified in FIG. 10. The view also indicates the severity 1114 of the dental finding. The patient can review the information provided along with the color-coded 3D virtual model 1112 and either proceed with the recommended treatment by selecting the accept 1108 icon or request additional information by selecting the request consult 1110 icon.

While the invention has been described in terms of specific aspects, it is apparent that other forms could be adopted by one skilled in the art. For example, the methods described herein could be performed in a manner which differs from the aspects described herein. The steps of each method could be performed using similar steps or steps producing the same result but which are not necessarily equivalent to the steps described herein. Some steps may also be performed in different orders to obtain the same result. Similarly, the apparatuses and systems described herein could differ in appearance and construction from the aspects described herein, the functions of each component of the apparatus could be performed by components of different constructions but capable of a similar though not necessarily equivalent function, and appropriate materials could be substituted for those noted. Accordingly, it should be understood that the invention is not limited to the specific aspects described herein. It should also be understood that the phraseology and terminology employed above are for the purpose of disclosing the illustrated aspects and do not necessarily serve as limitations to the scope of the invention.

What is claimed is:

1. A system for dental treatment planning, the system comprising:
an input module configured to receive and provide access to dental scan data of a dentition of a patient;
a 3D visualization module configured to generate a 3D virtual model of the dentition based on the dental scan data;
a report generation module configured to generate a radiology report based on the 3D virtual model, the radiology report identifying at least one dental finding;
a treatment planning module configured to generate a treatment plan, the treatment plan including at least one treatment option corresponding to the at least one dental finding; and
a communication module configured to present the 3D virtual model, the radiology report, and the treatment plan to the patient via a graphical user interface, the graphical user interface comprising user controls that facilitate interactive navigation and review of the 3D virtual model, the radiology report, and the treatment plan by the patient.

2. The system of claim 1, wherein the at least one dental finding and the at least one treatment option are represented within the 3D virtual model, and wherein the graphical user interface includes a data structure that is linked in data connection with and visually associated with the at least one dental finding and the at least one treatment option.

3. The system of claim 2, further comprising user-selectable icons selected from at least one of:
a first user-selectable icon in a first color, in data connection with the data structure, indicating no defect at the at least one dental finding;
a second user-selectable icon in a second color, in data connection with the data structure, indicating a moderate defect at the at least one dental finding; or
a third user-selectable icon in a third color, in data connection with the data structure, indicating a severe defect at the at least one dental finding.

4. The system of claim 1, wherein the user controls are responsive to user input that enables the patient to view and manipulate the 3D virtual model, the user controls selected from at least one of zooming, rotating, panning, or annotating specific areas of interest.

5. The system of claim 1, wherein the user controls are responsive to user input that enables the patient to select the at least one dental finding and access additional information associated with the at least one dental finding.

6. The system of claim 1, wherein the user controls are responsive to user input that enables the patient to select the at least one treatment option in the treatment plan and access additional information associated with the at least one treatment option, the additional information including a detailed description of the at least one treatment option, an estimated cost of the treatment option, and/or a timeline associated with the at least one treatment option.

7. The system of claim 1, wherein the 3D virtual model comprises a color-coded 3D virtual model to visually represent different data layers of the dentition.

8. The system of claim 1, wherein the 3D virtual model, the radiology report, and the treatment plan are presented to the patient as an integrated report via the graphical user interface, the integrated report comprising interactive elements that link the 3D virtual model with the at least one dental finding and the at least one treatment option.

9. The system of claim 1, wherein the 3D virtual model, the radiology report, and the treatment plan are communicated to the patient via an email containing a link to a secure patient portal.

10. The system of claim 1, further comprising a scheduling module in data communication with the communication module for enabling the patient to schedule an appointment with a dentist, via the graphical user interface, based on the treatment plan.

11. The system of claim 1, wherein:
the report generation module is configured to generate the radiology report based on input from a radiologist; and
the treatment planning module is configured to generate the treatment plan based on input from a dentist.

12. The system of claim 7, wherein the 3D visualization module further comprises:
a 3D model generation sub-module configured to generate a 3D model of the dental scan data;
a segmentation sub-module configured to identify and highlight individual dental structures within the 3D model; and,
an annotation sub-module configured to apply color-coded annotations to the highlighted individual dental structures to generate the color-coded 3D virtual model.

13. The system of claim 1, further comprising a payment processing module in data communication with the communication module for receiving payment from the patient for viewing and/or implementing the treatment plan.

14. The system of claim 1, wherein the user controls comprise at least one of:
tools for at least one of rotating, zooming, panning, and resetting a view of the 3D virtual model;
interactive 3D manipulation tools enabling the patient to at least one of click, drag, and rotate the 3D virtual model to view it from various angles;
dropdown menus or tabs for selecting the at least one dental finding and the at least one treatment option;
clickable elements to enable interaction with the treatment plan or accessing additional information; or
highlighting or annotation tools for emphasizing areas of interest on the 3D virtual model or providing patient feedback.

15. A non-transitory computer-readable medium having instructions stored thereon which, when executed by at least one computer processor, cause the at least one computer processor to:
receive and provide access to dental scan data of a dentition of a patient;
generate a 3D virtual model of the dentition based on the dental scan data;
generate a radiology report based on the 3D virtual model, the radiology report including at least one dental finding;
generate a treatment plan, the treatment plan including at least one treatment option corresponding to the at least one dental finding; and
present the 3D virtual model, the radiology report, and the treatment plan to the patient via a graphical user interface, the graphical user interface comprising user controls that facilitate interactive navigation and review by the patient.

16. The non-transitory computer-readable medium of claim 15, wherein the at least one dental finding and the at least one treatment option are represented within the 3D virtual model, and wherein the graphical user interface includes a data structure that is linked in data connection with and visually associated with the at least one dental finding and the at least one treatment option.

17. The non-transitory computer-readable medium of claim 16, further comprising instructions for displaying user-selectable icons selected from at least one of:
a first user-selectable icon in a first color, in data connection with the data structure, indicating no defect at the at least one dental finding;
a second user-selectable icon in a second color, in data connection with the data structure, indicating a moderate defect at the at least one dental finding; or
a third user-selectable icon in a third color, in data connection with the data structure, indicating a severe defect at the at least one dental finding.

18. The non-transitory computer-readable medium of claim 15, wherein the user controls are responsive to user input that enables the patient to at least one of:
view and manipulate the 3D virtual model, the user controls selected from at least one of zooming, rotating, panning, or annotating specific areas of interest;
select the at least one dental finding in the radiology report and access additional information associated with the at least one dental finding; or
select the at least one treatment option in the treatment plan and access additional information associated with the at least one treatment option, the additional information including a detailed description of the at least one treatment option, an estimated cost of the treatment option, and/or a timeline associated with the at least one treatment option.

19. The non-transitory computer-readable medium of claim 15, wherein the at least one computer processor is further configured to schedule an appointment with a dentist, via the graphical user interface, based on the treatment plan.

20. The non-transitory computer-readable medium of claim 15, wherein the 3D virtual model comprises a color-coded 3D virtual model to visually represent different data layers of the dentition; and wherein the at least one computer processor is further configured to:
generate a 3D model of the dental scan data;
identify and highlight individual dental structures within the 3D model; and
apply color-coded annotations to the highlighted dental structures to generate the color-coded 3D virtual model.
